(19) United States
(12) Patent Application Publication
(10) Pub. No.: US 2019/0224363 A1
(43) Pub. Date: Jul. 25, 2019

(54) INDICATOR DRESSING AND METHOD FOR INDICATING A CONTAMINATION, IN PARTICULAR OF A WOUND

(71) Applicant: Sefar AG, Heiden (CH)

(72) Inventors: Gerd GERDES, Abtwil (CH); Jérémie WEBER, Klaus (AT); Marc PREYSCH, Romanshorn (CH)

(73) Assignee: Sefar AG, Heiden (CH)

(21) Appl. No.: 16/330,728

(22) PCT Filed: Aug. 17, 2017
(86) PCT No.: PCT/EP2017/070835

§ 371 Date: Mar. 5, 2019

(30) Foreign Application Priority Data
Sep. 8, 2016 (EP) 16187791.5

(51) Int. Cl. A61L 15/56; A61B 5/00; A61L 15/38; A61L 15/42; G01N 33/52

(52) U.S. Cl. CPC A61L 15/56; A61B 5/6833; A61L 2300/442; A61L 15/42; G01N 33/528; A61L 15/38

(57) ABSTRACT

The invention relates to an indicator dressing with a carrier element, which is, at least in regions, provided with a marker material, which material, in a specific microbial contamination, changes a color. According to the invention, a first marker material is provided, which, in a first specific microbial contamination, changes color, and at least one second marker material is provided which changes color in a second specific microbial contamination, which material differs from the first microbial contamination. The invention moreover includes a method for indicating a contamination with this indicator dressing.

The invention relates to an indicator dressing with a carrier element, which is provided with a marker material, at least in regions, which marker material changes color in a specific microbial contamination, according to the preamble of claim 1.

The invention further relates to a method for indicating a contamination, in particular of a wound, wherein an indicator dressing, with a marker material, is placed upon the wound, wherein, in a specific microbial contamination, the color of the marker material changes, according to the preamble of claim 12.

A composition is known from WO 2000/024438 A1, which composition can indicate a microbial contamination on medical devices and materials. This indicator includes a substance or a substance composition, which, depending upon a presence of a microbial environment, can lead to a change in color. It is thereby signaled to medical personnel that a contamination is given or has at least reached a certain level.

Moreover, providing such type of indicator substances in wound dressings, as well, is known. A hint about a wound development can thereby be specified to the medical personnel in a timely manner. In particular, the usefulness or necessity of a changing of the wound dressing based on reaching of a certain contamination level can be displayed through a corresponding indicator. Further information cannot be derived from such a wound dressing.

The object underlying the invention is to disclose an indicator dressing and a method for indicating a contamination, in particular of a wound, by means of which a more comprehensive information about the contamination of the wound is enabled.

The object is on the one hand achieved by an indicator dressing having the features of claim 1 and, furthermore, by a method having the features of claim 12. Preferred embodiments of the invention are specified in the respectively dependent claims.

The indicator dressing according to the invention is characterized in that a first marker material is provided which, in a first specific microbial contamination, changes color, and that at least a second marker material is provided, which changes color in a second specific microbial contamination, which differs from the first microbial contamination.

The basic idea of the invention lies with providing the indicator dressing with at least two different marker materials, which materials, in different states or types, react to a microbial contamination. For example, a first marker material can display a slight contamination through discoloring, while the other marker material only reacts to a stronger contamination and discolors. This can give additional information to medical personnel that, for example, the inflammation of a wound has increased. Alternatively or additionally, it can be also provided that the different marker materials react to different types of contaminants, so that information about the type of contamination is indicated through the indicator dressing. The greater the number of marker materials, the more differentiatedly and exactly a contamination can be indicated.

The indicator dressing according to the invention is preferably employed on wounds of a human or animal patient. The indicator dressing can, however, also be used for other purposes, such as in medical devices or instruments, for which information about a degree or a type of the contamination is essential for the use thereof.

The different marker materials, which are provided in a quantity of two or more, can basically be arranged as distributed uniformly or over the entire surface the indicator dressing. In this case, the different marker materials cause different changes in color, so that the indicated color gives information, in particular about the type or the extent of the contamination. It is particularly preferable, according to one embodiment of the invention, that the first marker material is arranged on the carrier element distanced to the at least second marker material. The marker materials thus do not overlap and can offer an unambiguous indication. The marker materials can be applied upon the planar carrier element on one or two sides.

The marker materials are preferably physically separated, e.g. provided on separate base carriers, which are arranged or employed on the carrier element. The base carriers can be stand-alone indicator elements which differ in material, form, size, color, etc. These indicator elements are provided with the marker materials and are preferably arranged or attached on the carrier element separated via separating elements.

According to the further development of the indicator dressing according to invention, it is advantageous that the carrier element comprises a placement side for placing, in particular on a wound and an upper side, which is facing away from the placement side, and that a color can be discerned of the marker materials on the upper side. The carrier element can here be constructed in a mono or multi-layered fashion. The marker materials are here preferably located in an upper region of the carrier element, e.g. a covering element, wherein the marker materials are in connection with the contaminated surface, on to which the indicator dressing is applied. The arrangement of the marker materials here is selected such that a change in color is reliably discernible at the upper side.

According to a further development of the invention, an advantageous embodiment is that the carrier element comprises a transparent protective layer on the upper side. An outer protective layer is formed for the marker materials by the transparent layer. This increases the longevity and functionality of the indicator dressing.

The carrier element can basically be a perforated film or otherwise planar element. It is particularly advantageous according to a further development of the invention that the carrier element comprises at least one fabric, in particular a monofilament fabric. This is in particular biocompatible and is thus particularly gentle in the usage of the indicator dressing as a wound dressing.

The placement side is preferably formed by a biocompatible monofilament fabric out of monofilament threads, preferably with a smooth surface. On the monofilament fabric, a covering element is applied on to an upper side facing away from the dressing or the wound, which covering element is provided with the marker material.

The covering element with the marker material thusly is not directly in contact with the injured tissue of the wound. Rather, a separating layer is provided between the wound and the covering element with the marker materials, which layer is preferably formed from a biocompatible monofilament fabric. The monofilament fabric is formed from fine wire-like monofilament yarns. The covering element is preferably configured with the marker materials such that this element only reacts as a separating layer in an specific contamination behind the monofilament fabric. The covering element can receive the marker materials in solid, fluid and/or paste-like form.

In addition, an irritation of the wound by the provided marker materials is counteracted through the separating layer. Such types of interactions can lead to a disruption of the wound healing.

The carrier element is preferably formed from a fabric which is not irritating, in particular is not cytotoxic, is pyrogen-free, and is hypoallergenic.

The biocompatible monofilament fabric is preferably configured so stably that it serves as a support layer on which at least one covering element is firmly applied. This covering element itself can here comprise a single layer or multiple layers. Corresponding to the intended use of the indicator dressing, the covering element can additionally be configured in any desired way, e.g. to absorb wound secretion or to apply means promoting wound healing, e.g. an ointment or a fluid. The covering element can comprise a fabric, a non-woven material and/or another material, which is capable of receiving the marker materials.

A preferred embodiment of the invention is that the monofilament fabric is configured as a simple fabric with an open-mesh structure, which has a pore size from 5 μm to 500 μm, preferably 20 μm to 300 μm, and that the pores form a percentage of 15% to 17% of the area of the monofilament fabric. The monofilament fabric according to the invention is, according to this embodiment, thus particularly finely porous so that, while on the one hand an air and water penetration through the pores is possible, on the other hand, an adhesion or encompassing of the monofilament tissue through the fabric material is hardly possible, however. A protective but permeable indicator dressing, in particular a wound dressing, is thus created, which moreover ensures a clear delimiting and thusly an easy peel-off from a wound, however. The good permeability for air and fluid is particularly provided by that the pores form a percentage from 15% to 70% of the area of the monofilament fabric, preferably 40% to 70%. In this way, a high permeability is achieved.

Preferably, the carrier element is formed from fibers out of PA, PET, PP, PVDF and/or PTFE. The fibers, in particular monofilament fibers, have a diameter size between 20 to 500 μm, preferably between 30 to 150 μm. In a simple embodiment, the indicator dressing in provided with two different marker materials. Particularly good information is, according to a further embodiment variant of the invention, thereby achieved, that more than two different marker materials are provided.

Different marker materials are to be understood as being materially different or being differently configured or arranged in a different concentration or in another way in such a manner that these materials react in different types and/or degrees of contamination.

The marker materials can essentially be provided planarly on the carrier element, so that an outer side of the indicator dressing discolors over a relatively large surface. A particularly good indication of different contaminations is thereby achieved, according to a further development of the invention, in that the material markers are applied in the form of letters, numbers, patterns and/or symbols. In particular, the letters, numbers, patterns and/or symbols can be selected such that these give direct information about the intensity or type of the contamination. E.g. line or point patterns can be provided as patterns. E.g. the designations of infectious contaminants can be written out fully or in abbreviations with the marker material, as well. Thus, e.g. in a contamination through e-coli bacteria, the word “e-coli” can appear, or a symbol can refer to the already printed word.

In the usage of such manner of characters, the marker materials can change into the same color. It is particularly preferred, however, if the different marker materials also lead to a different discoloration.

The marker materials can essentially be applied, in any desired way, on the carrier element or on a layer of the carrier element. It is particularly preferred, according to an embodiment variant, that the marker materials are applied through screen printing, spray printing, or stamping. These methods are also particularly suited for applying fluid or paste-like marker materials in the form of letters, numbers, patterns, or symbols.

A further preferred embodiment of the invention is that an adhesive layer is provided, at least in regions, on a placement side of the carrier element. The adhesive layer here is usually an adhesive material application, which is suited for applying, in particular onto skin. The adhesive layer here can be applied directly onto the placement side.

Essentially, any suitable material can be used as marker material, which serves as an indicator for indicating a contamination state, in particular of a microbial wound environment, and in particular a contamination of a wound. It is particularly preferable, according to a further development of the invention, that the marker material includes at least one colorant, in particular a reactive colorant, a fluorescent colorant, a pH-sensitive substance, enzyme, cells, a hydrogel, a temperature-sensitive substance, a salt, an ointment and alginates. These materials are here selected such that, in a corresponding contamination environment, e.g. through degradation products of the bacteria, they lead to a change in color.

Moreover, it is provided, according to an advantageous embodiment variant of the invention, that the marker materials are colorless in an initial state or have the same color, wherein a discoloring occurs upon reaching of respectively specific contamination. In particular, such type of marker materials can be employed, in conventional wound applications, so that these applications are not perceived as disturbing. The carrier element preferably has a neutral color, e.g. white, while the marker materials change themselves to a contrasting color, e.g. red, blue, green, etc.

The method according to invention is characterized in that an indicator dressing according to the invention is placed-on so that, in a first microbial contamination, the color of a first marker material changes, and that, in a second microbial contamination, the color of a second marker material changes, wherein the first microbial contamination differs from the second microbial contamination.

The method according to the invention can be employed, in particular, in wounds of a human or animal body, but can also be employed in objects, e.g. medical devices and instruments. The already described advantages result in the employment of an indicator dressing according to the invention.

According to a further development of the method according to the invention, it is provided that the different marker materials react to a different degree of the microbial contamination and/or in case of different microbial contaminants. In particular, the marker material can be selected such that these react, respectively, to different contaminants.

A particularly good contamination indication is achieved, according to a further development of the method according to the invention, in that the marker materials are colorless in an initial state or have the same color, and discolor upon reaching of the respectively specific microbial contamination.

It is furthermore advantageous that the first marker material and the at least second marker material, upon reaching of the specific microbial contamination, assume a first color or at least one second color, respectively, which are different. Through the change into different colors, which contamination is present particularly easily clarified for medical personnel.

The invention is further explained below by means of a preferred exemplary embodiment, which is schematically represented in the drawings. The drawings show in:

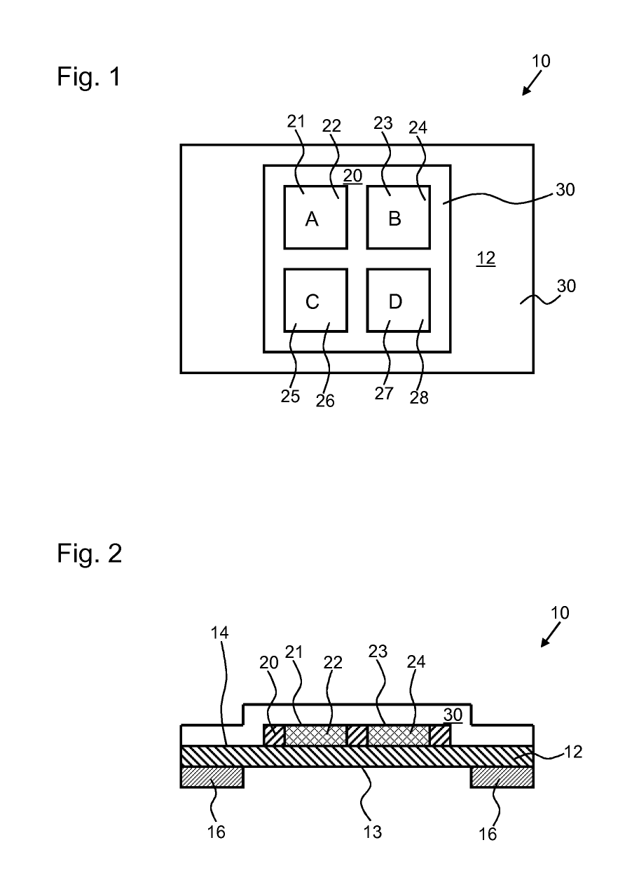

FIG. 1 a schematic plan view onto an indicator dressing according to the invention, and

FIG. 2 a schematic, cross-sectional view of the indicator dressing of FIG. 1, which is not true to scale.

FIGS. 1 and 2 illustrate an indicator dressing 10, which comprises a rectangular carrier element 12. The carrier element 12 is configured flexibly and as a biocompatible microfilament fabric. Two strip-shaped adhesive layers 16 are laterally provided on an placement side 13, which is provided for application upon a wound. A single adhesive layer 16 here consists of an adhesive material application, with which the indicator dressing can be releasably fastened e.g. along the edge of a wound.

The flexible carrier element 12 is provided with pores or openings so that a connection of the wound environment on the placement side 13 to a planar covering element 20 exists, which element is applied on an upper side 14 of the carrier element 12. The covering element 20 can likewise be configured as a fabric, in particular as a monofilament fabric, or a non-woven material or a coating element which is suitable for receiving marker materials.

A first region 21, a second region 23, a third region 25 and a fourth region 27 are arranged in the covering element 20, arranged spatially distanced from each other in the illustrated exemplary embodiment. The regions 21, 23, 25, 27 are provided with different marker materials, namely a first marker material 22, a second marker material 24, a third marker material 26 or a fourth marker material 28, respectively. The marker materials 22, 24, 26, 28 are, in the illustrated example, applied as fluids, and have penetrated the covering element 20. Here, the marker materials 22, 24, 26, 28, are arranged spatially distanced in the corresponding regions 21, 23, 25, 27, however. A separating means, e.g. a lacquer, can be provided in the interstitial spaces, in order to reliably delimit the individual regions 21, 23, 25, 27 from one another.

The marker materials 22, 24, 26, 28 here are selected to be different, so that these materials lead, in a respectively pre-specified different contamination, to a change in color. A change to a different color, respectively, preferably results. A labelling is provided, in the represented indicator dressing 10, in each region 21, 23, 25, 27, for the further improved signaling of a contamination, which labelling is schematically represented through the letters A, B, C or D. The labelling can also directly refer to the type of infectious contaminants. If a change in color results in the first region 21 with the first marker material 22, it is signaled, upon a change in color in the first region 21, to the medical personnel that an infection with the contaminant A is present.

The carrier element 12 with the covering element 20 is provided, on the upper side 14, with a transparent protective layer 30. This layer protects the covering element with the marker materials 22, 24, 26, 28 against external influences, but continues to allow to discern the change in color in the regions 21, 23, 25, 28.

1.-15. (canceled)

16. An indicator dressing for placing upon a wound, with a carrier element, which is provided, at least in regions, with marker materials, which, in a specific microbial contamination, change their color,
wherein
a placement side of the carrier element upon placement on to the wound is formed by a biocompatible monofilament fabric of monofilament threads,
the marker materials are applied in the form of letters and/or numbers,
a first marker material is provided, which, in a first specific microbial contamination, changes color, and
at least a second marker material is provided, which changes color in a second specific microbial contamination, which differs in type or amount from the first microbial contamination.

17. The indicator dressing according to claim 16,
wherein
the first marker material is arranged on the carrier element distanced to the at least second marker material.

18. The indicator dressing according to claim 16,
wherein
the carrier element comprises a placement side for placement, in particular on a wound, and an upper side which faces away from the placement side, and
in that a color of the marker materials can be discerned on the upper side.

19. The indicator dressing according to claim 18,
wherein
the carrier element comprises, on the upper side, a transparent protective layer.

20. The indicator dressing according to claim 16,
wherein
more than two different marker materials are provided.

21. The indicator dressing according to claim 16,
wherein
the letters and/or numbers, in the form of which the marker materials are applied, give direct information on the intensity or type of contamination.

22. The indicator dressing according to claim 16,
wherein
the marker materials are applied through screen printing, spray printing or stamping.

23. The indicator dressing according to claim 16,
wherein
the monofilament threads of the monofilament fabric of the carrier element are formed with a smooth surface.

24. The indicator dressing according to claim 16,
wherein
an adhesive layer is provided, at least in regions, on a placement side of the carrier element.

25. The indicator dressing according to claim 16,
wherein
the marker material includes at least one colorant, in particular a reactive colorant, a fluorescent colorant, a pH-sensitive substance, enzymes, cells, a hydrogel, a temperature-sensitive substance, a salt, an ointment, and/or alginates.

26. The indicator dressing according to claim 16,
wherein
the marker materials are colorless in an initial state, or have the same color, wherein a discoloring occurs upon reaching of the respectively specific contamination.

27. A method for indicating a contamination, in particular of a wound, wherein an indicator dressing with different marker materials is placed-on, wherein the color of the marker materials changes in a specific microbial contamination,
wherein
the indicator dressing according to claim 16 is placed-on,
in a first microbial contamination, the color of a first marker material changes, and
in a second microbial contamination, the color of a second marker material changes, wherein the first microbial contamination differs from the second microbial contamination,
wherein the changed marker materials indicate letters and/or numbers on the carrier element.

28. The method according to claim 27,
wherein
the different marker materials react to a different degree of microbial contamination and/or in different microbial germs.

29. The method according to claim 27,
wherein
the marker materials are colorless in an initial state, or have the same color and, upon achieving the respective specific microbial contamination, discolor.

30. The method according to claim 27,
wherein
the first marker material and the at least second marker material, upon achieving the specific microbial contamination, assume a first color or at least one second color, which are different.
